(19) United States
(12) Patent Application Publication
(10) Pub. No.: US 2006/0235269 A1
(43) Pub. Date: Oct. 19, 2006

(54) Method, apparatus and kit for bile or pancreatic duct endoscopy

(75) Inventor: Irving Waxman, Chicago, IL (US)

Correspondence Address: MICHAEL BEST & FRIEDRICH, LLP, ONE SOUTH PINCKNEY STREET, P O BOX 1806, MADISON, WI, 53701, US

(73) Assignee: The University of Chicago, Chicago, IL (US)

(21) Appl. No.: 11/107,056

(22) Filed: Apr. 15, 2005

(51) Int. Cl. A61B 1/00

(52) U.S. Cl. 600/104; 600/114; 600/128

(57) ABSTRACT

A method, apparatus and kit for performing bile or pancreatic duct endoscopy. In some embodiments, the kit includes a wire adapted to be navigated perorally or pernasally to a position within the biliary tree or the pancreatic duct. The kit can further include an endoscope having a generally tubular body movable along the wire to and within the biliary tree or pancreatic duct. The kit can further include a tool dimensioned to be received within and movable along the tubular body of the endoscope. In some embodiments, the apparatus includes the wire, the endoscope and an overtube positioned over the endoscope and movable along the wire. Also, in some embodiments the method includes navigating the wire to within the biliary tree or pancreatic duct, and installing the endoscope over the wire to a position within the biliary tree or pancreatic duct.

### BACKGROUND

Peroral cholangioscopy and percutaneous transhepatic cholangioscopy (PTCS) are existing nonsurgical procedures that permit direct visualization of the biliary tree and target biopsies of pathological findings. In peroral cholangioscopy, a small caliber (e.g., about 3 mm) scope (often referred to as a “baby” scope) is typically fed through the channel of a duodenoscope (“mother” scope) and is then advanced into the common bile duct (CBD). Overall, the procedure can be safer and faster than PTCS. However, such conventional peroral cholangioscopy procedures are generally expensive, time consuming, cumbersome, and require two experienced endoscopists: one to operate the mother scope, the other to operate the baby scope. Also, the lack of dedicated water and/or air channels compromises visibility and requires the assembly and use of a cumbersome water irrigation system, which can add time and effort to such procedures. Additional limitations include the extreme fragility of the baby scope (resulting in a short useful life of the baby scope and high repair costs), and the small caliber (e.g., less than about 2 mm) of the working channel that only permits passage of small biopsy forceps able to obtain very small and, in many cases, inadequate tissue samples. In light of these drawbacks, the popularity of peroral bile or pancreatic duct endoscopy has remained limited.

### SUMMARY

Some embodiments of the present invention provide a kit for bile or pancreatic duct endoscopy, wherein the kit comprises a wire adapted to be navigated perorally or pernasally to the ampulla of Vater; and an endoscope comprising a generally tubular body, the tubular body having a lumen and an outer diameter, the outer diameter being less than about 7 mm, the lumen dimensioned to receive the wire, the endoscope movable along the wire via relative movement between the lumen and the wire within the lumen for navigation of the endoscope along the wire to and within the ampulla of Vater.

In some embodiments, a kit for bile or pancreatic duct endoscopy is provided, and comprises an endoscope adapted to be navigated perorally or pernasally to at least one of the ampulla of Vater, the common bile duct, the pancreatic duct, the cystic duct, and the hepatic ducts, the endoscope comprising a generally tubular body, the tubular body including a lumen and an outer diameter, the outer diameter being less than approximately 7 mm; and a tool dimensioned to be received within the lumen of the tubular body of the endoscope, the tool adapted to perform at least one of diagnosis and therapy in at least one of the ampulla of Vater, the common bile duct, the pancreatic duct, the cystic duct, and the hepatic ducts.

Some embodiments of the present invention provide an apparatus for performing bile or pancreatic duct endoscopy upon a patient having a mouth and an ampulla of Vater, wherein the apparatus comprises a wire adapted to extend from the patient's mouth to the patient's ampulla of Vater; an endoscope comprising a generally tubular body, the tubular body including a lumen and an outer diameter, the outer diameter being less than about 7 mm, the lumen dimensioned to receive the wire such that the endoscope is positioned over and is movable along the wire; and an overtube having a lumen dimensioned to receive the endoscope, the overtube being positioned over the endoscope and movable along the wire.

In some embodiments, a method for performing endoscopy of a bile or pancreatic duct of a patient is provided and comprises navigating a wire perorally or pernasally to and within at least one of the ampulla of Vater, the common bile duct, the pancreatic duct, the cystic duct, and the hepatic ducts of the patient; providing an endoscope, the endoscope comprising a generally tubular body, the tubular body including a lumen and an outer diameter, the outer diameter being less than about 7 mm; installing the endoscope over the wire while the wire is in place within at least one of the ampulla of Vater, the common bile duct, the pancreatic duct, the cystic duct, and the hepatic ducts of the patient such that the wire is received within the lumen of the tubular body of the endoscope; navigating the endoscope along the wire to a position within at least one of the ampulla of Vater, the common bile duct, the pancreatic duct, the cystic duct, and the hepatic ducts; and viewing the at least one of the ampulla of Vater, the common bile duct, the pancreatic duct, the cystic duct, and the hepatic ducts with the endoscope.

Other features and aspects of the invention will become apparent by consideration of the detailed description and accompanying drawings.

### BRIEF DESCRIPTION OF THE DRAWINGS

FIG. 1 is a view of a bile or pancreatic duct endoscopy apparatus according to an embodiment of the present invention;

FIG. 1A is a close-up cut-away view of a portion of the bile or pancreatic duct endoscopy apparatus of FIG. 1;

FIG. 1B is a close-up cut-away view of another portion of the bile or pancreatic duct endoscopy apparatus of FIG. 1;

FIGS. 2-7 are schematic illustrations of various portions of the bile or pancreatic duct endoscopy apparatus of FIGS. 1, 1A and 1B, shown at various points in a method for performing bile or pancreatic duct endoscopy according to an embodiment of the present invention;

FIG. 7B is a schematic cross-sectional illustration of a portion of the bile or pancreatic duct endoscopy apparatus illustrated in FIG. 7.

FIG. 8 illustrates component parts of a plurality of kits for performing bile or pancreatic duct endoscopy according to several embodiments of the present invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

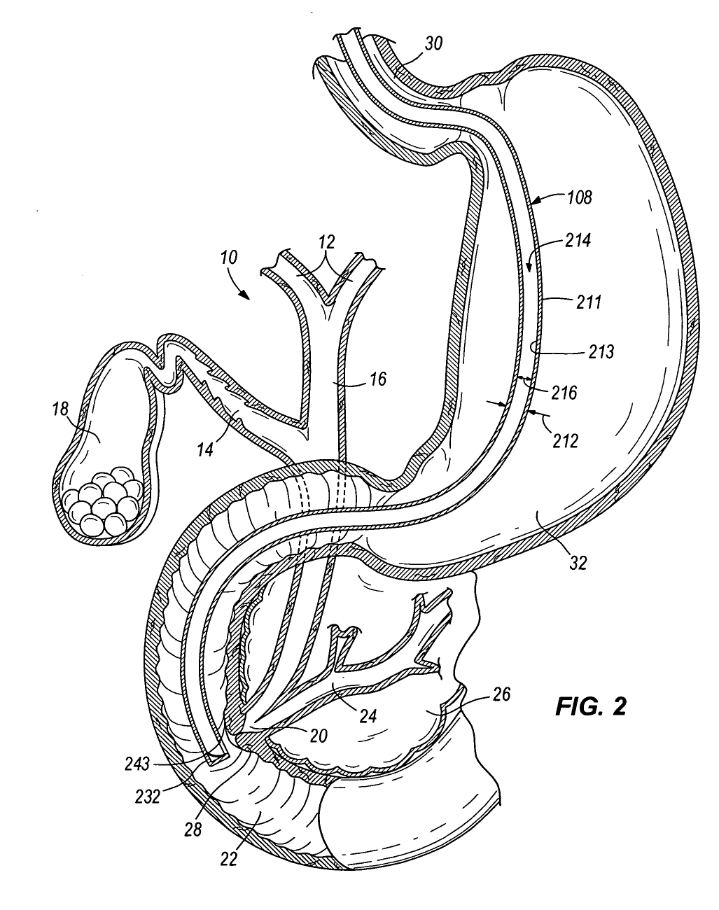

Also, as used herein and in the appended claims, the term “biliary tree” 10 (see FIGS. 2-7) collectively refers to the gallbladder 18 and the ducts through which bile travels, including the hepatic ducts 12 that carry bile from the liver, the cystic duct 14 that carries bile between the common bile duct 16 and the gallbladder 18, the common bile duct 16 that carries bile from the hepatic ducts 12 and/or the cystic duct 14, and the ampulla of Vater 20 (or the hepatopancreatic duct 20) that carries bile from the common bile duct 16 to the duodenum 22.

In some patients, the ampulla of Vater 20 also serves as the junction between the common bile duct 16 and the main pancreatic duct 24 (see FIGS. 2-7), as shown in FIGS. 2-7, and is therefore sometimes referred to as the “hepatopancreatic duct” 20. However, in some patients, the ampulla of Vater 20 serves merely as a termination of the common bile duct 16, and the pancreatic duct 24 enters the duodenum separately, next to the ampulla of Vater 20. Therefore, as used herein and in the appended claims, the term “ampulla of Vater” 20 is intended to encompass the common duct or swelling extending from the juncture of the common bile and pancreatic ducts (in some patients) as well as to either or both ducts extending from the common bile duct and pancreas, respectively. Although the ampulla of Vater 20 illustrated in the accompanying figures serves to carry bile and pancreatic juice to the duodenum 22, it should be understood that this structure need not carry pancreatic juice to the duodenum 22. Furthermore, the endoscopy methods, apparatuses and kits disclosed herein can be used to investigate the pancreatic duct separately by accessing the pancreatic duct 24 directly from the duodenum 22.

As used herein and in the appended claims, the term “bile duct endoscopy” refers to endoscopy of any portion of the biliary tree 10 of a patient. Similarly, as used herein and in the appended claims, the term “pancreatic duct endoscopy” refers to endoscopy of the main pancreatic duct 24 or smaller pancreatic ducts that feed the main pancreatic duct 24 (collectively referred to herein as “the pancreatic duct 24”). The main pancreatic duct 24 connects the pancreas 26 to the duodenum 22 directly or indirectly (e.g., via the ampulla of Vater 20 as described above). Accordingly, as used herein and in the appended claims, the term “bile or pancreatic duct endoscopy” refers to endoscopy of at least a portion of any one or more of the following: the ampulla of Vater 20, the pancreatic duct 24 (whether accessed indirectly via the ampulla of Vater 20 or directly via the duodenum 22), the common bile duct 16, the cystic duct 14, and the hepatic ducts 12.

Also, as used herein and in the appended claims, the terms “upstream” and “downstream” refer generally to the overall direction of fluid movement for a given, nonpathological biological process. That is, the term “upstream” is used to describe any location, element or process that occurs prior to the point or area being referred to relative to the direction of fluid movement, whereas the term “downstream” is used to describe any location, element or process that occurs subsequent to the point or area of reference with respect to fluid movement. For example, and with reference to FIGS. 2-7, bile flows from the liver via hepatic ducts 12 to the common bile duct 16, and to the ampulla of Vater 20. Accordingly, the hepatic ducts 12 are upstream of the common bile duct 16, and the common bile duct 16 is upstream of the ampulla of Vater 20; whereas, the ampulla of Vater 20 is downstream of the common bile duct 16, and the common bile duct 16 is downstream of the hepatic ducts 12.

In addition, as used herein and in the appended claims, the terms “proximal” and “distal” are used to refer to relative locations or positions with respect to an origin. That is, the term “proximal” is used to describe any location or position that is situated nearer the origin, whereas the term “distal” is used to describe any location or position that is situated farther from the origin. In the present invention, the origin is generally defined as the location or position of entry into an internal cavity of a patient (i.e., the position of the physician), and the terms “proximal” and “distal” are used to describe locations or positions relative to that entry point. For example, an endoscope has a proximal end that can be grasped by a physician, and a distal end that can be positioned within an internal cavity of a patient. The distal end of the endoscope can be moved distally in the internal cavity. For example, the distal end of the endoscope can enter the mouth, and then be moved distally into the esophagus, the stomach, and so forth, to capture images of the areas of interest within the patient.

Furthermore, as used herein and in the appended claims, the term “retrograde” refers to movement in a direction generally opposite the usual direction of movement or flow. For example, endoscopic retrograde cholangiography (ERC) includes navigating an endoscope from the duodenum 22 (see FIGS. 2-7), to the ampulla of Vater 20, to the common bile duct 16, and so forth, because the endoscope is being navigated opposite the direction that bile normally moves in these structures.

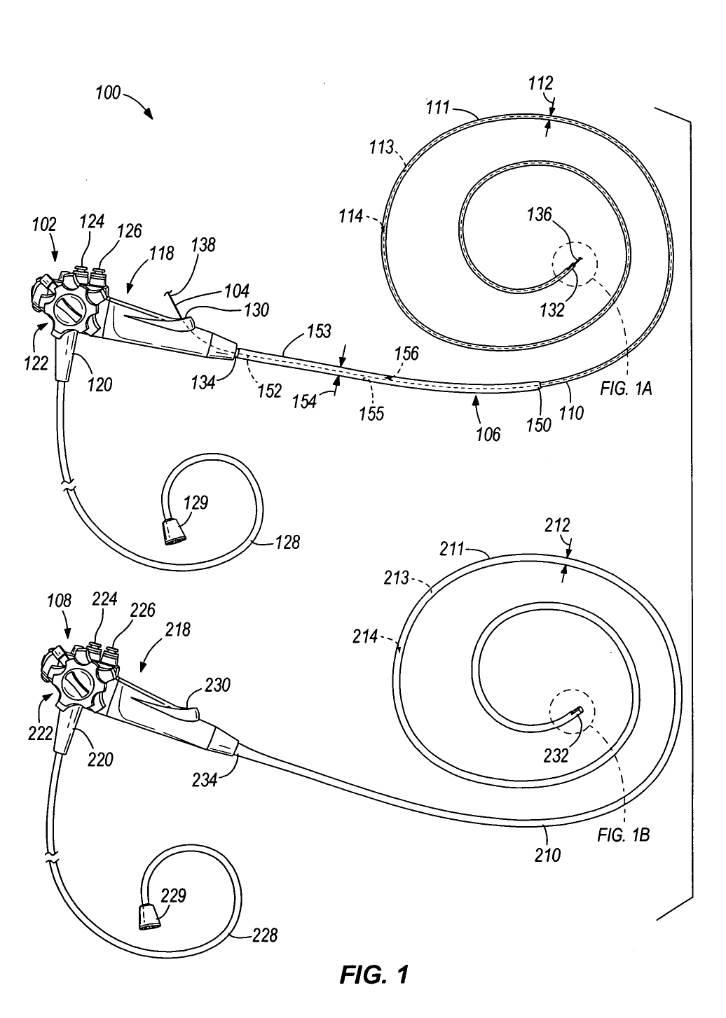

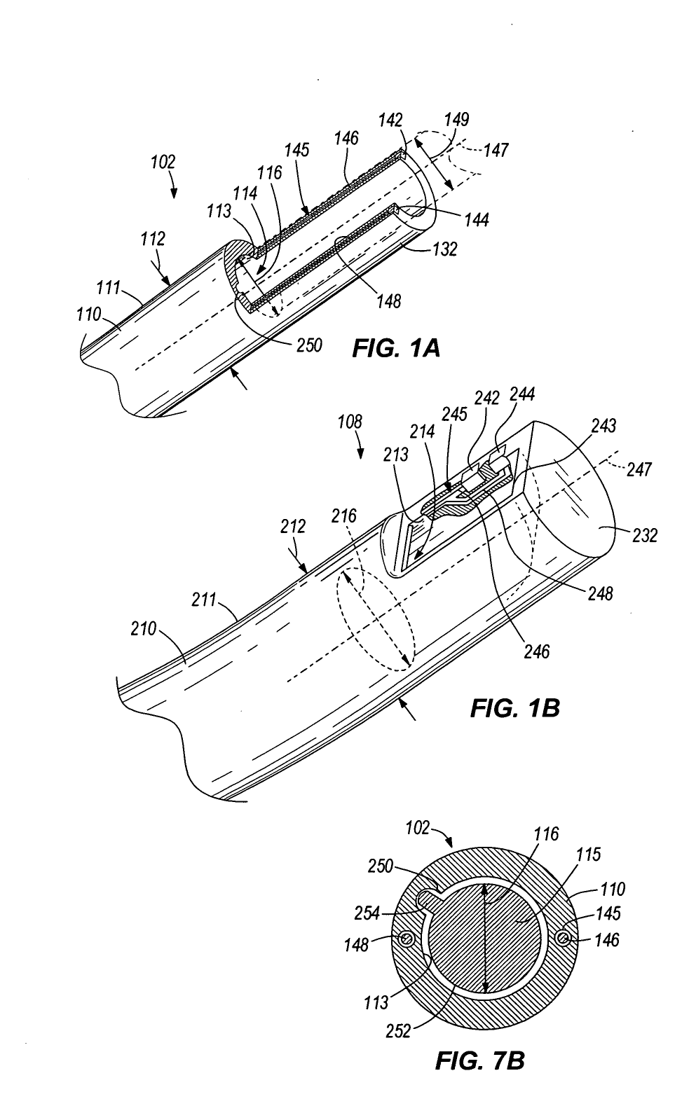

FIGS. 1, 1A and 1B illustrate a bile or pancreatic duct endoscopy apparatus 100 (also referred to herein as “endoscopy apparatus” 100) according to an embodiment of the present invention. In some embodiments, the endoscopy apparatus 100 includes a first endoscope 102, a wire 104, an overtube 106, and a second endoscope 108.

The first endoscope 102 can include a generally tubular body 110 shaped and dimensioned to be received in an internal cavity of a patient's body, and specifically, in the biliary tree 10 and/or pancreatic duct 24 of the patient. The tubular body 110 of the first endoscope 102 in the illustrated embodiment has an outer surface 111 that defines an outer diameter 112 and an inner surface 113 that defines a lumen 114 through the tubular body 110 and an inner diameter 116. As used herein and in the appended claims, the term “diameter” is employed to refer to a thickest dimension of an element's cross-sectional shape, and does not alone indicate or imply the shape of the element.

The lumen 114 is sometimes referred to herein as a “working channel” 114. The outer diameter 112 can be sized to access a variety of locations within the biliary tree 10 and the pancreatic duct 24. In some embodiments, the outer diameter 112 is less than about 7 mm, which allows the tubular body 110 of the endoscope 102 to access the ampulla of Vater 20 and various regions within the biliary tree 10 or pancreatic duct 24. The entrance to the ampulla of Vater 20 is defined by the major duodenal papilla 28 and the sphincter of Oddi, a muscle that encircles the entrance to the ampulla of Vater 20. For many applications, the sphincter of Oddi will need to be cut (e.g., a 5-10 mm incision can be made) or dilated to allow the wire 104 and/or the tubular body 110 to access the ampulla of Vater 20.

In some embodiments, the outer diameter 112 of the tubular body 110 is less than about 6 mm, which can allow the tubular body 110 to access the common bile duct 16, and hepatic ducts 12 in normal or diseased conditions. Also, in some embodiments, the outer diameter 112 of the tubular body 110 is less than about 5 mm, which can allow the tubular body 110 to access the common bile duct 16, the hepatic ducts 12, and in some cases, the cystic duct 14 and the pancreatic duct 24 in normal or conditions. For example, in some embodiments, the outer diameter 112 of the tubular body 110 is between about 5 mm and about 6 mm (e.g., such as a 5.6 mm GIF-XP 160 ultra-slim upper endoscope, available from Olympus America, Inc., Melville, N.Y.). In some embodiments, the outer diameter 112 of the tubular body 110 is at least about 3 mm. Also, in some embodiments, the outer diameter 112 is at least about 4 mm. For example, in some embodiments the outer diameter 112 is greater than about 3.4 mm. The smaller the outer diameter 112, the further upstream the tubular body 110 will be allowed to navigate.

The above description relating outer diameter 112 of the tubular body 110 to accessible sites is given as a general approximation of the anatomical structures that may be accessed under normal or diseased conditions with an outer diameter 112 of these sizes. However, patient variability can affect which ducts are accessible by various sizes of tubular bodies 110. In addition, diseased or pathological conditions can affect which ducts are accessible by various sizes of tubular bodies 110. For example, under some pathological conditions, the pancreatic duct 24 may be accessible by a tubular body 110 having an outer diameter 112 of about 6 mm, and under some pathological conditions, the cystic duct 14 may be accessible by a tubular body 110 having an outer diameter of about 5 mm.

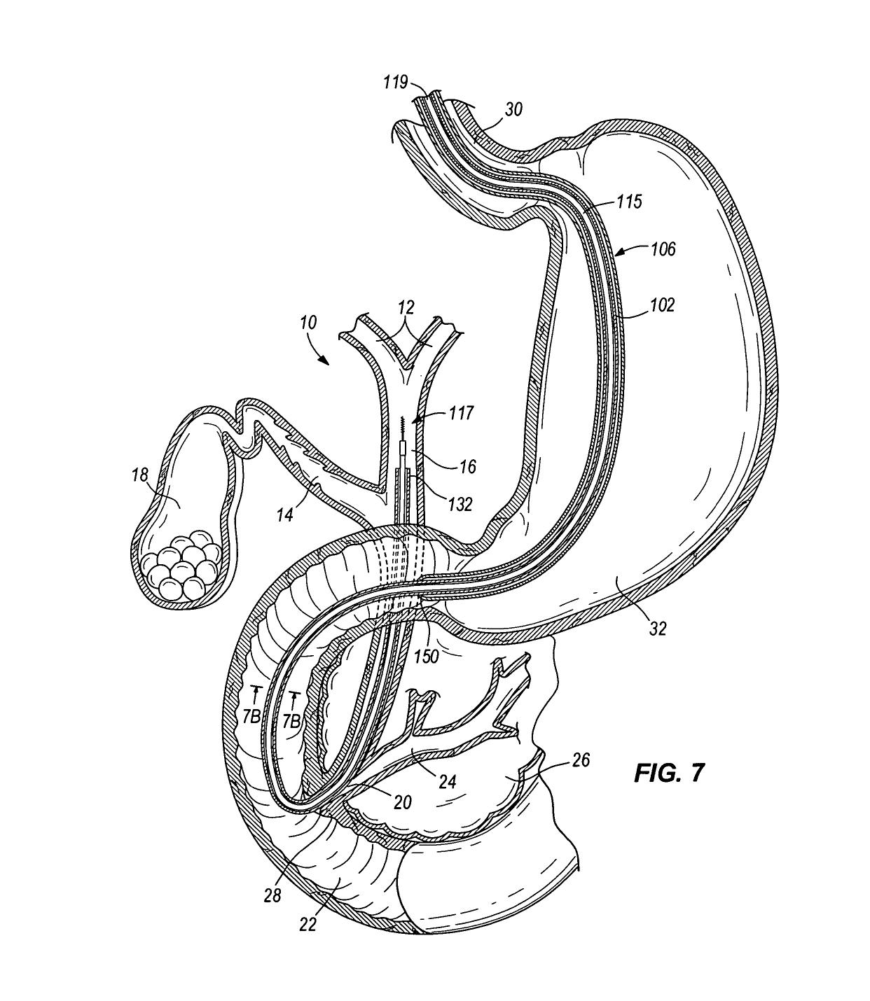

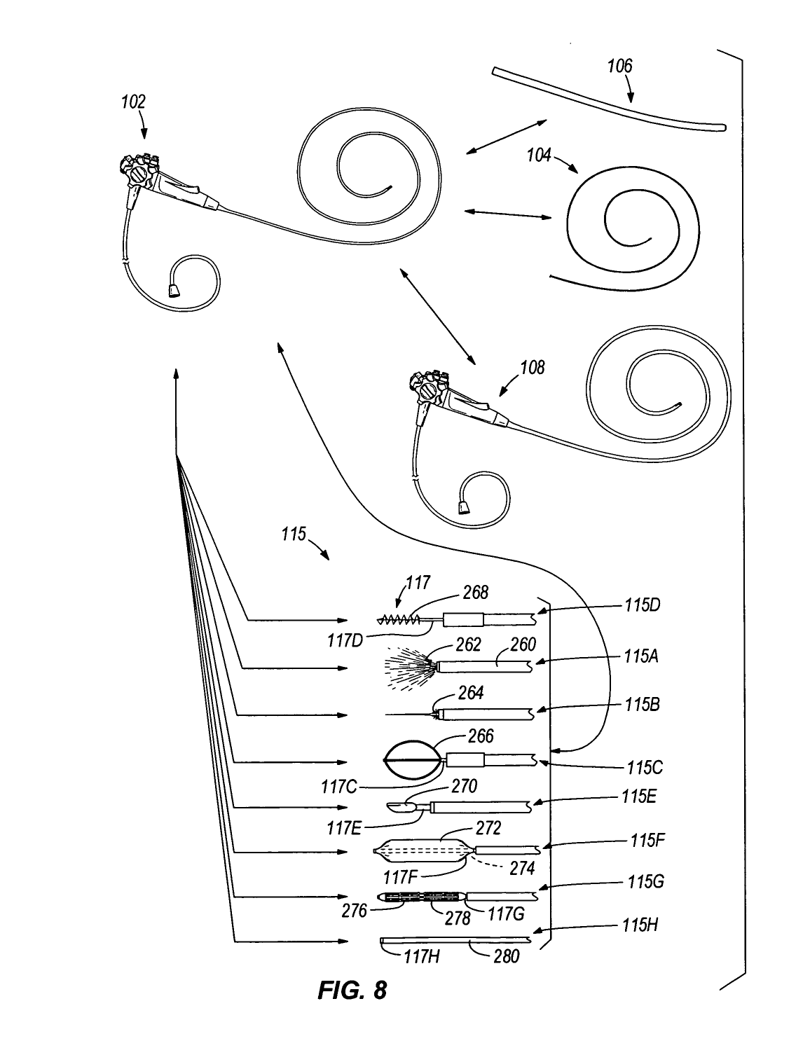

The inner diameter 116 of the tubular body 110 can be large enough to allow the wire 104 and/or one or more types of tools 115 (see FIGS. 7 and 8) to be received within and moved relative to the lumen 114. Each tool 115 can include a distal end 117 and a proximal end 119. In addition, the inner diameter 116 of the tubular body 110 can be sized to allow the tools 115 to be navigated within and along the lumen 114 to a variety of sites in the biliary tree 10 for a variety of diagnostic and therapeutic procedures, as described in greater detail below with reference to FIGS. 7 and 8, including sphincterotomy, lithotripsy, stone removal, stone reduction, tissue removal, stenting, cauterization, biopsy, surgery, and the like. In some embodiments, the inner diameter 116 is at least about 1.2 mm to allow a variety of diagnostic and therapeutic tools to fit within the lumen 114 of the tubular body 110, and be moved along the lumen 114. In some embodiments, the inner diameter 116 is at least about 2 mm. For example, a tubular body 110 having an inner diameter 116 no less than about 2 mm and no greater than about 6 mm can be utilized for tool insertion and movement, as well as for wire insertion and movement as described in greater detail below.

In some embodiments, the first endoscope 102 also includes a control portion 118. The control portion 118 can include a handle 120, a plurality of controls (e.g., dials 122 in the illustrated embodiment) for controlling the first endoscope 102 (e.g., for controlling flexion of the tubular body 110), an irrigation valve 124, a suction valve 126, and/or a cable 128 that houses power connection wiring, fiber optics wiring, and the like. The cable 128 can include a connector 129 allowing the cable 128 to be connected to a power supply, a computer or computer peripheral device (which in some embodiments can run cholangiography software or other instructions, can be microprocessor-based, and/or can include a database or other information repository), a monitor, dedicated video processing and/or viewing equipment, a fluid supply, a suction device, and/or other devices and systems for operation of the first endoscope 102. The control portion 118 can further include a lumen port 130 permitting access to the lumen 114 of the tubular body 110 of the first endoscope 102. In some embodiments, the lumen port 130 can allow other devices (e.g., the wire 104, one or more tools 115, and the like) to be moved into and along the lumen 114 and to be manipulated by the physician.

As shown in FIG. 1, the tubular body 110 includes a distal end 132 and a proximal end 134 in fluid communication with the lumen port 130. In addition, the wire 104 includes a distal end 136 and a proximal end 138. As also shown in FIG. 1, the tubular body 110 of the first endoscope 102 includes a length. The length can be dimensioned to allow the tubular body 110 to reach desired anatomical structures.

As described in greater detail below with reference to FIGS. 2-7, the wire 104 can be positioned within the internal cavities that are desired to be investigated, and the first endoscope 102 can be installed over the wire 104. In other words, when the distal end 136 of the wire 104 has been positioned within the desired internal cavity, the distal end 132 of the tubular body 110 can be slid over the proximal end 138 of the wire 104 and moved relative to the wire 104 to navigate the distal end 132 of the tubular body 110 to the desired location (e.g., adjacent the distal end 136 of the wire 104, in some embodiments), as shown in FIGS. 1 and 1A.

With continued reference to the illustrated embodiment, the distal end 132 of the tubular body 110 and the distal end 136 of the wire 104 are shown in greater detail in FIG. IA. The distal end 132 of the tubular body 110 of the first endoscope 102 can include a light 142 positioned to illuminate anatomical structures to be investigated as the distal end 132 is moved within an internal cavity of a patient. The distal end 132 can also include a camera 144 for viewing and/or recording images of anatomical structures as the distal end 132 is moved along the internal cavity of a patient.

The light 142 and the camera 144 are shown in the illustrated embodiment as being positioned in a space 145 defined between the outer surface 111 and the inner surface 113 of the tubular body 110. The light 142 and the camera 144 are also illustrated as being substantially flush with the distal end 132 of the tubular body 110. However, the light 142 and/or the camera 144 can be recessed or protrude with respect to the surrounding portions of the tubular body 110, in other embodiments. Also with reference to the illustrated embodiment, the lumen 114 can extend through the distal end 132 of the tubular body 110 in a direction substantially parallel to a longitudinal axis 147 of the tubular body 110. In addition, the light 142 and the camera 144 in the illustrated embodiment are positioned to direct light and retrieve images, respectively, in a direction substantially parallel to the longitudinal axis 147 of the tubular body 110. Accordingly, the illustrated embodiment of the first endoscope 102 is an end-viewing endoscope. In other embodiments, the lumen can be in fluid communication with the exterior of the tubular body 110 via one or more apertures in any other portion of the distal end 132 of the tubular body 110. In this regard, any other type of endoscope can be used (e.g., side-viewing endoscopes, and the like) without departing from the present invention.

Power supply wiring 146 and fiber optics wiring 148 can be connected to the light 142 and the camera 144 and can extend through the space 145 of the tubular body 110 along at least a portion of the length of the tubular body 110. The locations, orientations and configurations of the light 142, camera 144, power supply wiring 146, and fiber optics wiring 148 are shown in FIG. 1A by way of example only. Any other arrangement, configuration and relative orientation of these elements can be employed as desired without departing from the spirit and scope of the present invention.

In some embodiments, the wire 104 includes appropriate dimensions and properties (e.g., length, outer diameter 149, shape, degree of taper (if any), stiffness, flexibility, elasticity, biocompatibility, and the like) that allow the wire 104 to be navigated perorally or pernasally to the biliary tree 10 or pancreatic duct 24. A variety of wires 104 can be used for this purpose. For example, in some embodiments, the wire 104 includes a 0.035-inch JAGWIRE® Super Stiff guidewire available from Boston Scientific, Natick, Mass. As shown in FIG. 1, in some embodiments the outer diameter 149 of the wire 104 gradually tapers from the proximal end 138 to the distal end 136. In other embodiments, this taper can be more or less pronounced, and can extend along any portion of the length of the wire 104. Also, in some embodiments the distal end 136 of the wire 104 has an outer diameter 149 of less than or equal to about 0.035 inches (0.889 mm) to allow the wire 104 to readily navigate past the major duodenal papilla 28 to the ampulla of Vater 20.

In some embodiments, the wire 104 is stiffer than the 0.035-inch JAGWIRE® Super Stiff guidewire. In some embodiments, the wire 104 includes a lumen through which a removable core can be positioned and moved relative to the wire 104 to provide greater stiffness to at least a portion of the length of the wire 104. In some embodiments, the distal end 136 (e.g., the distal 3-5 cm of the wire 104) has different properties (e.g., reduced stiffness) than the remainder of the length of the wire 104, because different material properties are needed for navigating into tiny, fragile structures than are needed for supporting an endoscope or for navigating through large anatomical structures. For example, navigating the first endoscope 102 through the stomach 32 can cause the first endoscope 102 to curl up, wind, tangle or loop in the stomach 32, instead of continuing on a substantially straight and direct path through the stomach 32. The wire 104 can assist in tracking the first endoscope 102 through the stomach 32, and can minimize looping of the first endoscope 102 in the stomach 32 by allowing the first endoscope 102 to be navigated over the wire 104.

The overtube 106 can be used to assist in navigating the first endoscope 102 along the wire 104 to reach desired locations within the patient. Overtubes 106 that can be used in conjunction with the present invention include overtubes supplied by Fujinon Photo. The overtube 106 includes a distal end 150 and a proximal end 152. The overtube 106 can also include an outer surface 153 that defines an outer diameter 154 sized to allow the overtube 106 to be inserted perorally or pernasally. The overtube 106 can also include an inner surface 155 that defines a lumen 156 through the overtube 106 and an inner diameter 158. As shown in FIG. 1, the inner diameter 158 can be dimensioned to allow the first endoscope 102 to be received within and navigated along the lumen 156 of the overtube 106, and in some embodiments to allow the first endoscope 102 to pass through the overtube 106. That is, the inner diameter 158 of the overtube 106 is greater than the outer diameter 112 of the first endoscope 102, in some embodiments. In order to reduce the opportunity for interference between the overtube 106 and internal cavities of the patient, the distal end 150 of the overtube 106 can be rounded or chamfered, or can otherwise be shaped without sharp edges or corners.

As shown in FIGS. 2-7, in some applications the wire 104 can be inserted perorally or pernasally into a patient's esophagus 30 and can be navigated via the stomach 32 to the duodenum 22 and biliary tree 10 or pancreatic duct 24, such as by another endoscope that can then be removed from the patient (as described in greater detail below). As mentioned above, the first endoscope 102 can loop in the stomach 32, and the wire 104 can minimize looping of the first endoscope 102 in the stomach 32. In addition, because the wire 104 is small relative to the volume of the stomach 32, installing the first endoscope 102 over the wire 104 in the stomach 32 can be difficult and can cause the wire 104 and/or the first endoscope 102 to loop in the stomach 32 as the first endoscope 102 is advanced along the wire 104 in the stomach 32. Accordingly, in some embodiments, the overtube 106 can be installed over the wire 104 (e.g., by backloading the overtube 106 over the wire 104 prior to installing the first endoscope 102 over the wire 104) and positioned within the stomach 32 to guide the first endoscope 102 and to substantially prevent the wire 104 and/or the first endoscope 102 from looping as the first endoscope 102 is moved along the wire 104 in the stomach 32.

The overtube 106 illustrated in FIGS. 1-8 has a length that is less than the length of the tubular body 110 of the first endoscope 102. Specifically, as shown in FIGS. 2-7, the length of the overtube 106 allows the overtube 106 to be navigated to a position adjacent a distal portion of the stomach 32 and a proximal portion of the duodenum 22 (e.g., the duodenal bulb). However, in some embodiments, the length of the overtube 106 is sized to allow the overtube 106 to be navigated further through the duodenum 22 to a position adjacent the entrance to the ampulla of Vater 20 (and the entrance to the pancreatic duct 24, in some patients). The overtube can have a number of different lengths and diameters for receiving the first endoscope 102 and for assisting in navigating the first endoscope 102 along the wire 104 in relatively large internal cavities. In some embodiments, the overtube 106 includes an inner diameter 158 that is greater than the outer diameter 112 of the tubular body 110 of the first endoscope 102, and that allows the tubular body 110 to move relative to the lumen 156 of the overtube 106 without friction. For example, in some embodiments, the overtube 106 includes an inner diameter of at least about 8 mm. In some embodiments, the overtube 106 includes an outer diameter of less than about 1 cm. In some embodiments, the overtube 106 includes a length of at least about 50 cm to readily access a distal portion of the stomach 32 or a proximal portion of the duodenum 22.

With continued reference to FIG. 1, the second endoscope 108 in the illustrated embodiment shares many of the same elements and features described above with reference to the first endoscope 102, wherein like numerals represent like elements. Accordingly, elements and features of the second endoscope 108 corresponding to elements and features of first endoscope 102 are provided with the same reference numerals in the 200 series. Reference is made to the description of the first endoscope 102 above for a more complete description of the features and elements (and alternatives to such features and elements) of the second endoscope 108.

In some embodiments, the second endoscope 108 includes a tubular body 210 having an outer surface 211 that defines an outer diameter 212, an inner surface 213 that defines a lumen 214 and an inner diameter 216, and a control portion 218. The tubular body 210 can include a length that extends from a distal end 232 to a proximal end 234, and can have a lumen port 230 in fluid communication with the lumen 214.

The outer and inner diameters 212, 216 and the cross-sectional lumen size of the tubular body 210 of the second endoscope 108 can be generally larger than outer and inner diameters 112, 116 and the cross-sectional lumen size of the tubular body 110 of the first endoscope 102. As shown in FIG. 2, the second endoscope 108 is adapted to be navigated perorally or pernasally into the esophagus 30, the stomach 32 and the duodenum 22 to a position adjacent the entrance to the ampulla of Vater 20 (and the pancreatic duct 24, in some patients). The second endoscope 108 in the illustrated embodiment is adapted to be navigated to such a position without requiring the assistance or guidance of the wire 104 or the overtube 106. However, the inner diameter 216 and the lumen 214 can be sized to allow the wire 104 to be received within and moved along the lumen 214 of the tubular body 210.

The distal end 232 of the tubular body 210 according to an embodiment of the present invention is shown in greater detail in FIG. 1B. As shown in FIG. 1B, the distal end 232 can include a light 242 positioned to illuminate anatomical structures to be investigated as the distal end 232 is moved along an internal cavity within a patient. The distal end 232 can further include a camera 244 for viewing and/or recording images of anatomical structures as the distal end 232 is moved along the internal cavity of the patient.

The light 242 and the camera 244 of the second endoscope 108 are shown by way of example in FIG. 1B as being positioned in a space 245 defined between the outer surface 211 and the inner surface 213 of the tubular body 210. The tubular body 210 can include an aperture 243 defined therein in fluid communication with the lumen 214 of the tubular body 110 to allow fluid and/or devices to be passed into and out of the lumen 214 via the aperture 243. The aperture 243 can be oriented in any manner with respect to a longitudinal axis 247 of the tubular body 210, and in the illustrated embodiment is oriented substantially orthogonally to the longitudinal axis 247 of the tubular body 210.

The light 242 and the camera 244 can be positioned adjacent the aperture 243, or in any other locations suitable for illuminating and capturing images of an area of interest within the patient's body. The light 242 and camera 244 are substantially flush with the outer surface 211 of the tubular body 110 in the illustrated embodiment, but could instead be recessed or protrude with respect to surrounding portions of the tubular body 210. With continued reference to the illustrated embodiment, the light 242 and the camera 244 are directed substantially orthogonally to the longitudinal axis 247 of the tubular body 210. Accordingly, the second endoscope 108 is a side-viewing endoscope. Employing a side-viewing endoscope can permit the second endoscope 108 to be navigated through the duodenum 22 to a position adjacent the ampulla of Vater 20 without tortuous twisting or turning of the distal end 232 of the tubular body 210 to locate the entrance to the ampulla of Vater 20. The location of the aperture 243 also allows for side access to the lumen 214 of the tubular body 210. It should be understood, however, that the second endoscope 108 can include an end-viewing endoscope without departing from the spirit and scope of the present invention.

In the illustrated embodiment (see FIG. 1B), the distal end 232 of the tubular body 210 is closed, and the lumen 214 of the second endoscope 108 does not extend through the distal end 232. However, in other embodiments, the lumen 214 does extend through the distal end 232 of the tubular body 210. Furthermore, in the embodiment shown in FIG. 1B, the aperture 243 is positioned at the distal end 232 of the tubular body 210. However, in other embodiments, the aperture 243 can be positioned anywhere along the length of the tubular body 210, and need not be located at or proximate the distal end 232. In addition, although the aperture 243 in the illustrated embodiment is positioned substantially orthogonally with respect to the longitudinal axis 247 of the tubular body 210 (to allow the lumen 214 to be accessed from a side of the tubular body 210), the aperture 243 can be positioned at a variety of angles or orientations relative to the longitudinal axis 247 of the tubular body 210 while still allowing fluid and/or devices to be moved into or out of a side of the lumen 214.

Power supply wiring 246 and fiber optics wiring 248 can be connected to the light 242 and the camera 244, and can extend through the space 245 of the tubular body 210 along any portion of the length of the tubular body 210. The locations, orientations and configuration of the light 242, camera 244, power supply wiring 246 and fiber optics wiring 248 are shown in FIG. 1B by way of example only. Any other arrangement, configuration and relative orientation of these elements can be employed as desired without departing from the spirit and scope of the present invention.

In some embodiments, the second endoscope 108 includes a duodenoscope, such as the TJF-160 duodenoscope, available from Olympus America, Melville, N.Y.

A bile or pancreatic duct endoscopy method (referred to herein as “the method”) according to an embodiment of the present invention is illustrated in FIGS. 2-7, and is presented by way of example only.

With reference first to FIG. 2, the method can include navigating the distal end 232 of the tubular body 210 of the second endoscope 108 perorally or pernasally into the esophagus 30 of a patient, into the stomach 32, and into the duodenum 22 to a position adjacent the entrance to the ampulla of Vater 20. The distal end 232 can be properly positioned by using the light 242 and the camera 244 of the second endoscope 108 to image the inner wall of the duodenum 22 to locate the entrance to the ampulla of Vater 20. When the entrance to the ampulla of Vater 20 is located, the aperture 243 of the second endoscope 108 can be positioned adjacent (e.g., at least partially in line) with the entrance to the ampulla of Vater 20.

As mentioned above, after the second endoscope 108 has been properly positioned within the duodenum 22, the sphincter of Oddi can be cut or dilated to allow or enhance access to the ampulla of Vater 20.

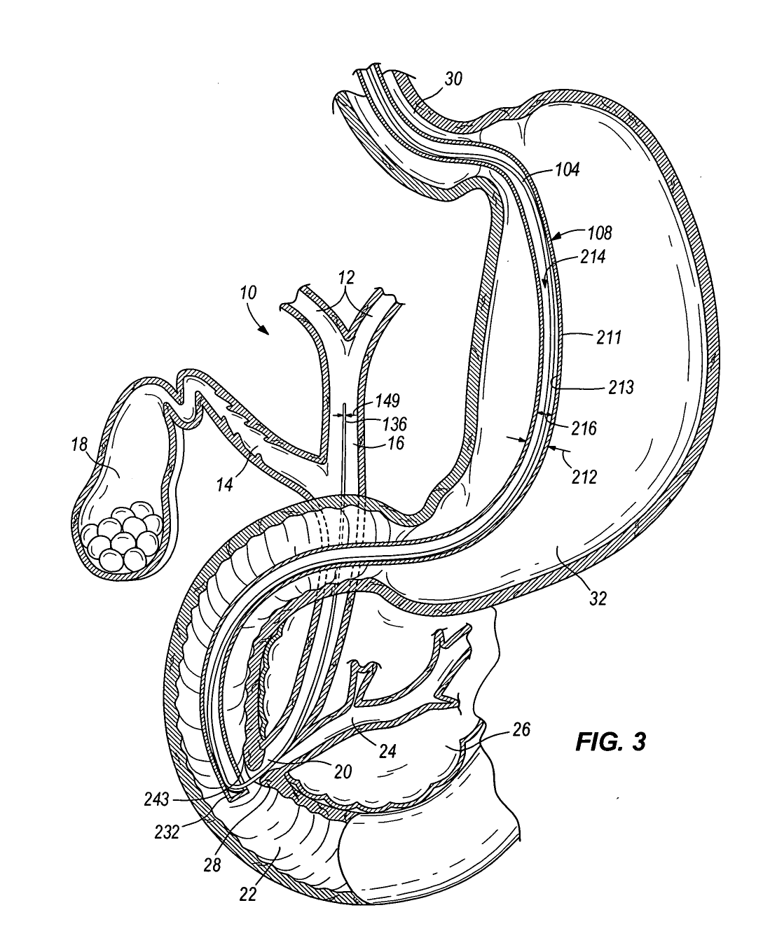

As shown in FIG. 3, after the second endoscope 108 has been properly positioned within the duodenum 22, the distal end 136 of the wire 104 can be navigated into the lumen port 230 of the second endoscope 108 and into the lumen 214 of the tubular body 210 of the second endoscope 108. The wire 104 can be moved along the lumen 214 of the tubular body 210 until the distal end 136 of the wire 104 reaches the aperture 243 in the tubular body 210. In other embodiments, the wire 104 can be placed in this position with respect to the second endoscope prior to insertion and navigation of the second endoscope 108 within the patient as described above. The distal end 136 of the wire 104 can then be navigated out of lumen 214 via the aperture 243, past the major duodenal papilla 28, and into the ampulla of Vater 20 (or into the entrance to the pancreatic duct 24, in some patients). The distal end 136 of the wire 104 can be further navigated upstream (i.e., in a retrograde manner) to at least one of the common bile duct 16, the pancreatic duct 24, the cystic duct 14, and the hepatic ducts 12. By way of example only, the distal end 136 of the wire 104 is illustrated in FIG. 3 as being positioned within the common bile duct 16.

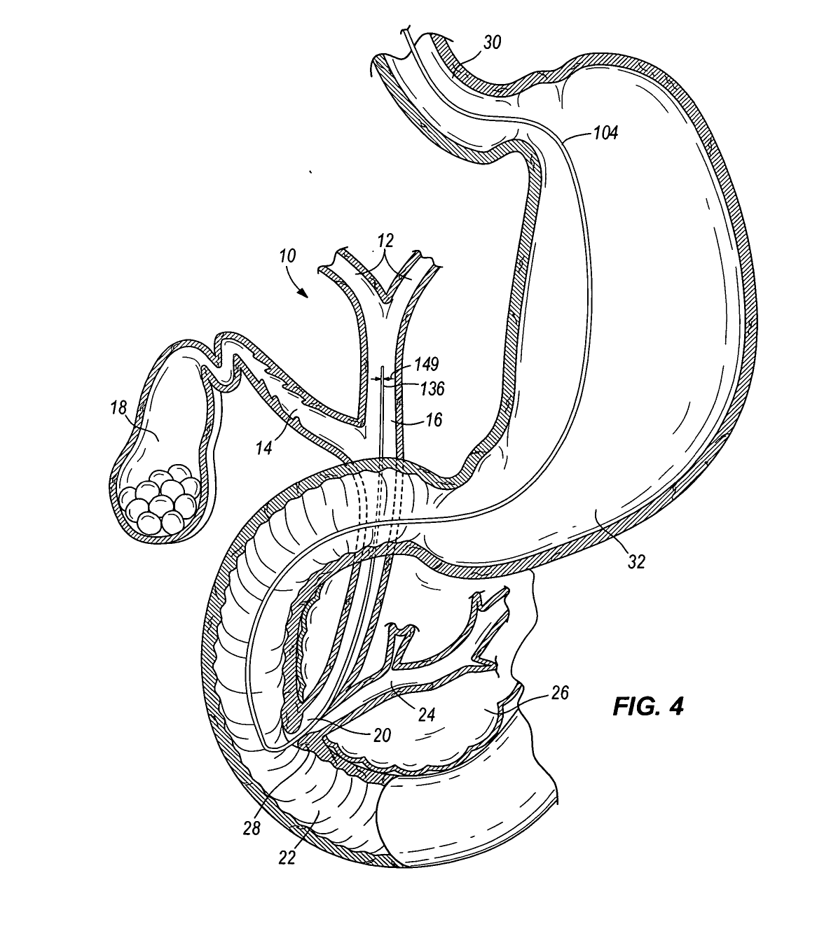

As shown in FIG. 4, the second endoscope 108 can be removed after the wire 104 has been positioned in the desired location within the biliary tree 10 or the pancreatic duct 24. The second endoscope 108 can be removed by moving the second endoscope 108 proximally relative to the wire 104 so that the lumen 214 of the second endoscope 108 moves relative to the wire 104, and the wire 104 remains positioned in the biliary tree 10 or pancreatic duct 24.

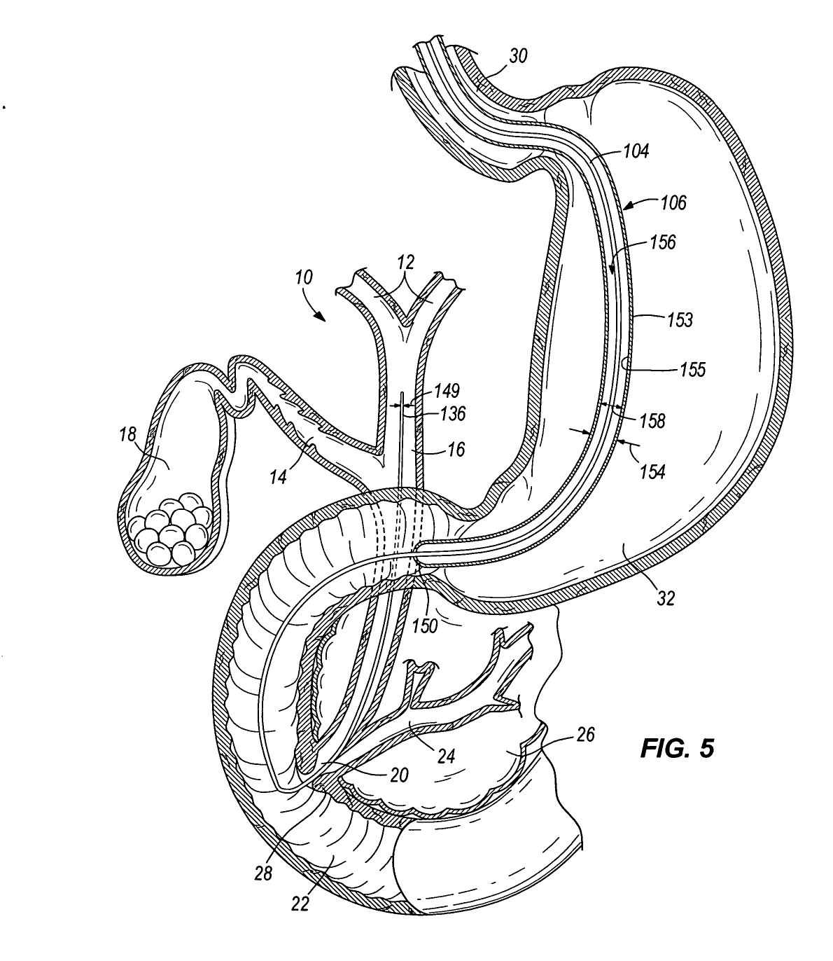

As shown in FIG. 5, in some embodiments the overtube 106 can be installed over the wire 104 by backloading the overtube 106 onto the wire 104. In other words, the rounded distal end 150 of the overtube 106 in the illustrated embodiment can be positioned over the proximal end 138 of the wire 104 and moved distally along and over the wire 104 such that the lumen 156 of the overtube 106 is moved relative to the wire 104. In the embodiment illustrated in FIG. 5, the length of the overtube 106 is less than that of the wire 104. Therefore, the overtube 106 is installed over a portion of the wire 104, and extends to a location adjacent a distal portion of the stomach 32, or a proximal portion of the duodenum 22. As mentioned above, in some embodiments, the length of the overtube 106 can be greater than that illustrated in FIG. 5, and/or the overtube 106 can be positioned to extend further distally than what is illustrated in FIG. 5. It should also be noted that the overtube 106 can be shorter than that shown in the illustrated embodiment.

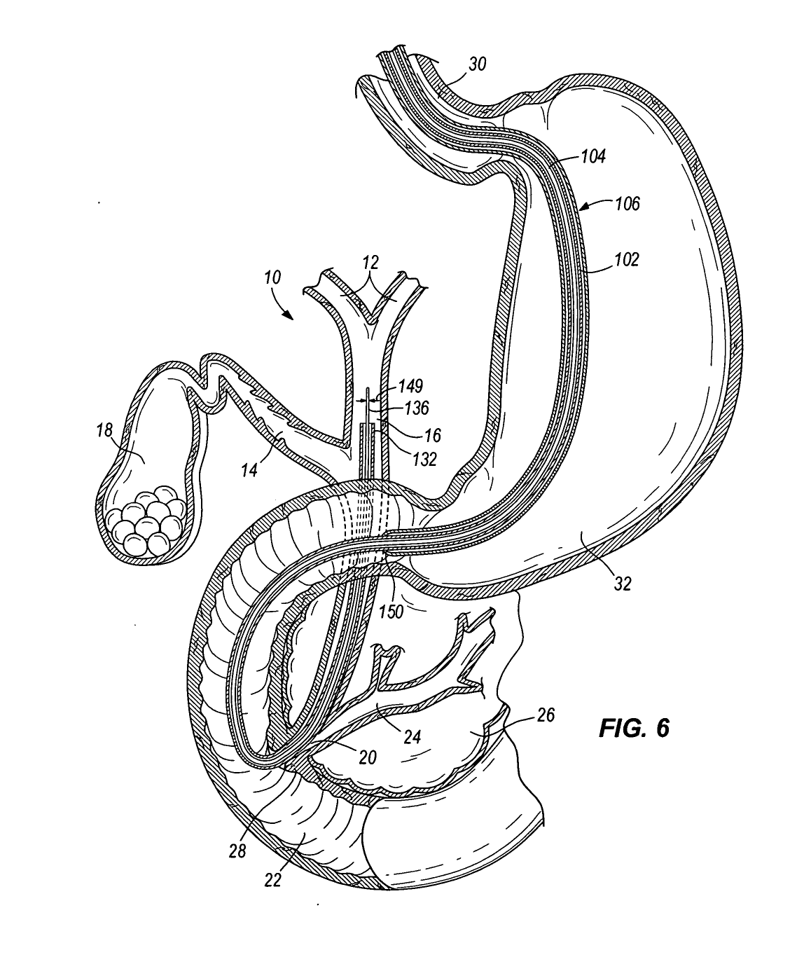

As shown in FIG. 6, in some embodiments the first endoscope 102 can be installed over the wire 104 by backloading the tubular body 110 of the first endoscope 102 over the wire 104. That is, the distal end 132 of the tubular body 110 can be positioned over the proximal end 138 of the wire 104 and can be moved distally along and over the wire 104 such that the lumen 114 of the tubular body 110 is moved relative to the wire 104. In some embodiments, the inner diameter 158 of the overtube 106 can be sized to allow the tubular body 110 of the first endoscope 102 to be received within and moved relative to the lumen 156 of the overtube 106, at least for a portion of the length of the wire 104 as shown in FIG. 6. Specifically, in the embodiment illustrated in FIG. 6, the tubular body 110 of the first endoscope 102 is navigated within and along the lumen 156 of the overtube 106 through the stomach 32. After the distal end 132 of the tubular body 110 reaches the rounded distal end 150 of the overtube 106 (i.e., in some embodiments, adjacent a distal portion of the stomach 32), the distal end 132 of the tubular body 110 exits the lumen 156 of the overtube 106 and continues along and over the wire 104 to the biliary tree 10 or the pancreatic duct 24. The distal end 132 of the tubular body 110 can be navigated to a position adjacent the distal end 136 of the wire 104. As illustrated in FIG. 6, for example, the distal end 132 of the tubular body 110 is navigated to a position within the common bile duct 16.

As shown in FIG. 7, in some embodiments the wire 104 can be removed from the lumen 114 of the tubular body 110 of the first endoscope 102. In some embodiments, removing the wire 104 can increase the amount of space within the lumen 114 available for other devices or tools 115. As shown in FIG. 7, one or more tools 115 can be positioned within the lumen 114 of the tubular body 110 by, for example, inserting the distal end 117 of the tool 115 into the lumen port 130 of the first endoscope 102. The distal end 117 of the tool 115 can then be moved along and relative to the lumen 114 of the tubular body 110 until it reaches the distal end 132 of the tubular body 110. In the embodiment shown in FIG. 7, for example, the distal end 117 is navigated to a position within the common bile duct 16.

In some embodiments, the overtube 106 (if employed) can be removed after the first endoscope 102 has been navigated to a desired location in the patient, or at least after the first endoscope 102 has passed through the overtube 106 and the location of the patient's body in which the overtube 106 is located.

As shown in FIG. 7B, the inner surface 113 of the tubular body 110 can include a recess 250 defined therein that extends along at least a portion of the tubular body 110, and in some embodiments extends along substantially the entire length of the tubular body 110. One or more tools 115 can be provided with at least one protrusion on an outer surface 252 thereof. The protrusion 254 can be shaped and dimensioned to be received within the recess 250 of the tubular body 110 such that movement of the tubular body 110 in a substantially circumferential direction (e.g., twisting of the tubular body 110) generates rotational movement of the tool 115. In the embodiment illustrated in FIG. 7B, the outer surface 252 of the tool 115 does not fit tightly within the lumen 114 of the tubular body 110. Rather, there is an amount of clearance between the outer surface 252 of the tool 115 and the inner surface 113 of the tubular body 110 in order to allow the tool 115 to be moved along and relative to the tubular body 110. In some embodiments, the first endoscope 102 can be adapted to receive at least one tool 115 within the lumen 114 of the tubular body 110 as just described. In such embodiments, the distal end 117 of the tool 115 can be moved into position by moving the tool 115 into the lumen 114 and along the tubular body 110 when the tool 115 is needed.

FIG. 7B illustrates the recess 250 as being defined in the inner surface 113 of the tubular body 110, and the protrusion 254 as extending from the outer surface 252 of the tool 115 by way of example only. It should be understood that the inner surface 113 can instead include a protrusion, and the outer surface 252 of the tool 115 can instead include a recess. Furthermore, only one mating recess 250 and protrusion 254 are illustrated in FIG. 7B. However, it will be appreciated that any number of additional mating features of the tool 115 and tubular body 110 can be utilized as desired. Furthermore, the protrusion 254 and recess 250 illustrated in FIG. 7B represent only one type of engagement between the tool 115 and tubular body 250.

A variety of other types of engagement can be established between the outer surface 252 of the tool 115 and the inner surface 113 of the tubular body 110 to allow the tool 115 to be manipulated by manipulating the tubular body 110 without departing from the spirit and scope of the present invention.

FIG. 8 illustrates a plurality of kits for performing bile or pancreatic duct endoscopy according to several embodiments of the present invention. The bile or pancreatic duct endoscopy apparatus 100 illustrated in FIG. 1 includes several component parts that can be combined in a variety of different manners for being packaged, marketed, and/or sold as kits. For example, as shown in FIG. 8, the first endoscope 102 can be combined with at least one of the overtube 106, the wire 104, the second endoscope 108, and one or more tools 115 to form a kit according to different embodiments of the present invention.

As further illustrated in FIG. 8, a variety of tools 115 can be used with the present invention to perform a variety of diagnostic and therapeutic procedures. The first endoscope 102 can be combined with one or more of these tools 115 to form different kits for performing a variety of diagnostic and therapeutic procedures. A variety of tools 115 (each including a unique distal end 117) is illustrated in FIG. 8 by way of example only. In this regard, it should be understood that a number of other tools can be used with the present invention, as known to those of ordinary skill in the art.

Packaging component parts of the bile or pancreatic duct endoscopy apparatus 100 in any of the kits disclosed herein can enable manufacturers to have increased control over the use of their products by physicians and others. In this regard, a manufacturer can encourage and provide instructions regarding the use of various combinations of products produced by the manufacturer and/or regarding the intended manner in which such products are to be used. With such control, the performance of products produced and sold by the manufacturer can increase significantly based at least upon the performance predictability of such products produced by the manufacturer.

By providing a manufacturer with increased control over which products are used in conjunction with other products, the likelihood of products being misused or mishandled (e.g., combined with unknown or untested third-party products, operated in unpredictable ways, and the like) or failing and/or resulting in injury or equipment damage can be reduced significantly. For example, a user may otherwise use a manufacturer's product in conjunction with an incompatible product manufactured by another party. Such use may cause the product to break or fail, may void the warranty of the manufacturer's product and/or the third-party product, and may result in unexpected problems during medical procedures. By packaging various products intended to be used together in a kit, the manufacturer can reduce the likelihood of these unfortunate events.

Furthermore, the kits disclosed herein can reduce the likelihood that poorly-founded warranty claims or other liability claims will be brought against the manufacturer, particularly in situations where a user has inappropriately used a manufacturer's product in a manner in which it was not intended (e.g., with incompatible products manufactured by others).

As shown in FIG. 8, a first tool 1 15A includes a fluid delivery or removal device 260. In some embodiments, the fluid delivery or removal device 260 can include a fluid passage 262 that can be connected to an irrigation and/or suction system to allow at least one of a liquid (e.g., water), gas (e.g., oxygen), or a combination of gases (e.g., air) to be delivered to and/or removed from a site of interest within the biliary tree 10 or the pancreatic duct 24. For example, following lithotripsy of a gallstone, the fragments or pieces of the gallstone should be removed to avoid blockage of any portion of the biliary tree 10 by the gallstone fragments or pieces. If the fragments or pieces are small enough, they can be removed by suction via the fluid delivery or removal device 260. As another example, during an endoscopic procedure, the area within the biliary tree 10 to be investigated may need to be flushed with water to better visualize the area (e.g., in order to clear bile covering surfaces of the area and inhibiting visualization of anatomical structures under investigation).

Also with reference to FIG. 8, a second tool 115B includes a laser 264, which can be operated at a particular wavelength and power for performing one or more types of treatments. For example, the laser 264 can be used to destroy tissue (e.g., endoscopic cauterization) or break up stones (e.g., lithotripsy). The laser 264 can include an Nd-YAG laser, a holmium laser, a tunable-dye laser, and combinations thereof.

As shown in FIG. 8, a third tool 115C includes a basket 266 positioned at its distal end 117C (e.g., a Dormia basket, available from Olympus America, Melville, N.Y.). The basket 266 can be used in therapeutic procedures, including, without limitation, procedures to entrap and remove stones (e.g., gallstones) or fragments of stones. The basket 266 can also be used in diagnostic procedures, including, without limitation, intraductal biopsy, where a portion of a stone, build-up, plaque, or other material within the biliary tree 10 can be removed and analyzed (e.g., for cancerous cells). Baskets 266 are well-known in the art and therefore not described further herein.

As shown in FIG. 8, a fourth tool 11 5D includes a brush 268 positioned at its distal end 117D (e.g., at least one of a cleaning brush, a cytology brush, and the like). The brush 268 can be used to clear out occluded areas within the biliary tree 10 or the pancreatic duct 24 either as a treatment or to better visualize an area of interest. The brush 268 can further be used for cell and/or tissue remove, or to obtain cytology samples (e.g., for identifying/distinguishing malignant tissue).

As shown in FIG. 8, a fifth tool 115E includes a blade 270 positioned at its distal end 117E. The blade 270 can be used for sphincterotomy (e.g., of the sphincter of Oddi), for biopsy, or for surgery. In addition, the blade 270 can be used to cut through an occlusion in the biliary tree 10 or pancreatic duct 24, to remove cells and/or tissue, or the blade 270 can be used in combination with a basket 266 or a balloon 272 (described below) to cut and remove an infected or suspicious portion of the biliary tree 10 or pancreatic duct 24 for further analysis and diagnosis.

As shown in FIG. 8, a sixth tool 115F includes a balloon 272 positioned at its distal end 117F (e.g., at least one of an extraction balloon for removing stones or stone fragments, a balloon dilator for stretching, thinning and/or narrowing stones or other occlusions, and combinations thereof). In some embodiments, for example, the balloon 272 can be positioned on a catheter 274 and in fluid communication with a lumen of the catheter 274. The lumen of the catheter 274 can be in fluid communication with a balloon inflating device external to the patient, as known in the art. The balloon 272 can be positioned within an occluded area of the biliary tree 10 or the pancreatic duct 24, and can be inflated to widen any portion of the biliary tree 10 or pancreatic duct 24 and/or to reduce the size of a stone, or force out plaque or other build-up within any portion of the biliary tree 10 or pancreatic duct 24. Furthermore, in some embodiments, the balloon 272 can be positioned upstream of a gallstone, inflated, and then moved downstream to pull the gallstone out of the biliary tree and into the duodenum 22.

A seventh tool 115G can be used alone or in combination with the sixth tool 115F described above. The seventh tool 115G includes a stent 276 positioned at its distal end 117G. In some embodiments, the stent 276 can be positioned on a catheter 278 in a reduced-diameter configuration. As known in the art, the stent 276 can be a balloon-expandable stent or a self-expanding stent 276. The stent 276 can be positioned in an occluded area within the biliary tree 10 or the pancreatic duct 24, and the diameter of the stent 276 can be increased to approximately an inner diameter of the duct to be treated. The stent 276 can thereby hold plaque or other build-up against an inner surface of the duct to remove blockage and increase the flow of fluid in the duct. In some embodiments, the stent 276 is placed after treatment with the balloon 272.

As shown in FIG. 8, an eighth tool 11 5H includes a fiber optic viewing device 280 at its distal end 117H. The fiber optic viewing device 280 can be used to view and/or obtain images of ducts that are too small to be navigated with the first endoscope 102. In such cases, the smaller fiber optic viewing device 280 can be extended distally from the distal end 132 of the tubular body 110 of the first endoscope 102 into narrow structures that the distal end 132 of the tubular body 110 cannot access.

The above description of tools 115 and the corresponding illustrations in FIG. 8 are meant to be illustrative of tools 115 that can be used with the present invention, and are not meant to be limiting. Other tools 115 can be used in accordance with the present invention, including, without limitation, at least one of biopsy forceps, retrieval forceps, retrieval loops, washing pipes, and the like.

Working examples of the present invention will now be described, are meant to be illustrative of the present invention, and do not indicate or imply a limitation upon the present invention.

In a first example, the second endoscope 108 included a standard side-viewing therapeutic duodenoscope (TJF-160, Olympus America, Inc., Melville, N.Y.); the wire 104 included a 0.035-inch diameter super stiff Jagwire (Boston Scientific Corp., Natick, Mass.); and the first endoscope 102 included an ultra-slim upper endoscope (GIF-XP 160, Olympus America). An overtube 106 was not used.

Endoscopic retrograde cholangiography (ERC) were performed upon patients using the standard side-viewing therapeutic duodenoscope. Following completion of the ERC procedure, a 0.035-inch diameter super stiff Jagwire 104 (Boston Scientific Corp., Natick, Mass.) was placed in the CBD 16. Using the wire 104 to maintain access, the duodenoscope 108 was removed and the wire 104 was back loaded on to an ultra-slim upper endoscope 102 (GIF-XP 160, Olympus America), which was advanced over the guidewire 104 under fluoroscopic and endoscopic control into the duodenum 22 and then across the ampulla of Vater 20 into the CBD 16 and upstream.

Direct cholangioscopy was attempted and successfully performed in 3 patients, as will now be described.

Patient No. 1 was a 79-year old male with a history of a para-ampullary choledocho-duodenal fistula and persistent/recurrent choledocholithiasis requiring multiple ERC in the past. This patient presented with cholangitis six weeks after the last ERC, during which a pigtail stent was left in place. An ERC was initially performed, and showed the presence of a pigtail stent across the para-ampullary fistula and purulent draining material. The stent was removed, and the CBD 16 cannulated through the orifice of the fistula. Contrast injection demonstrated an extremely dilated CBD 16 (20 mm) with upstream biliary dilatation and numerous filling defects in the CBD 16, CHD, and left hepatic duct 12. Multiple large and small muddy brown stones were extracted using a combination of an 18 mm extractor balloon 115F (Wilson-Cook) and a Dormia basket 115C (Olympus). Direct cholangioscopy as described above revealed the persistence of large amounts of sludge and stones, and an extremely dilated biliary system. Based upon these findings, surgical consultation to evaluate the possibility of performing an hepaticojejunostomy was recommended.

Patient No. 2 was a 91-year old female with a history of a large stone (about 1.5 cm) removed 6 weeks earlier, and presented for a follow-up ERC performed to remove the previously-placed biliary stent and evaluate the completeness of stones clearance. After removal of the biliary stent, the CBD 16 was cannulated and swept with a 15 mm extractor balloon 115F, resulting in retrieval of some sludge. With the wire 104 in place, the duodenoscope 108 was exchanged for the pediatric upper endoscope 108, which was advanced through the previously performed sphincterotomy site into the CBD 16. Cholangioscopy of the CBD 16, CHD, and left and right main biliary ducts was then performed and revealed a limited amount of sludge in the left biliary system, which was completely suctioned through the endoscope 108, obviating the need for repeat stent placement.

Patient No. 3 was a 79-year old female who presented with jaundice in the setting of choledocholithiasis, which was seen on abdominal computed tomography performed at admission. Using a sphincterotome (Boston Scientific), the major papilla was successfully cannulated, and contrast injection showed the presence of a single large stone in the distal CBD 16. A sphincterotomy was then performed, and an attempt at stone extraction with a Dormia basket 115C was made but failed. A mechanical lithotripsy basket was subsequently used to break the stone, and the resulting stone fragments were extracted using a 11.5 mm extractor balloon 115F. Using the same technique, cholangioscopy was performed, and revealed complete clearance of the CBD 16, CHD, and left and right biliary ducts.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

What is claimed is:

1. A kit for bile or pancreatic duct endoscopy, the kit comprising:
a wire adapted to be navigated perorally or pernasally to the ampulla of Vater; and
an endoscope comprising a generally tubular body, the tubular body having a lumen and an outer diameter, the outer diameter being less than about 7 mm, the lumen dimensioned to receive the wire, the endoscope movable along the wire via relative movement between the lumen and the wire within the lumen for navigation of the endoscope along the wire to and within the ampulla of Vater.

2. The kit of claim 1, wherein the lumen of the endoscope is defined by an inner diameter, and wherein the inner diameter is at least about 2 mm.

3. The kit of claim 1, wherein the endoscope is further adapted to be navigated via movement with respect to the wire from the ampulla of Vater to at least one of the pancreatic duct, the common bile duct, the cystic duct, and the hepatic ducts.

4. The kit of claim 1, further comprising an overtube having a lumen dimensioned to receive the endoscope.

5. The kit of claim 4, wherein the overtube is adapted to be positioned over the endoscope adjacent a portion of the wire.

6. The kit of claim 1, further comprising a tool dimensioned to be received within the lumen of the tubular body of the endoscope.

7. The kit of claim 6, wherein the tool includes at least one of a fluid delivery device, a fluid removal device, a laser, a basket, a brush, a blade, a balloon dilator, a balloon, a catheter, a stent, a fiber optic viewing device, and combinations thereof.

8. The kit of claim 1, wherein the endoscope is a first endoscope, the kit further comprising a second endoscope having a generally tubular body, the tubular body of the second endoscope having a second lumen having a cross-sectional area greater than that of the lumen of the tubular body of the first endoscope, the second lumen dimensioned to receive the wire to allow relative movement between the second lumen and the wire.

9. The kit of claim 1, wherein the outer diameter of the tubular body of the endoscope is greater than about 3.4 mm.

10. A kit for bile or pancreatic duct endoscopy, the kit comprising:
an endoscope adapted to be navigated perorally or pernasally to at least one of the ampulla of Vater, the common bile duct, the pancreatic duct, the cystic duct, and the hepatic ducts, the endoscope comprising a generally tubular body, the tubular body including a lumen and an outer diameter, the outer diameter being less than approximately 7 mm; and
a tool dimensioned to be received within the lumen of the tubular body of the endoscope, the tool adapted to perform at least one of diagnosis and therapy in at least one of the ampulla of Vater, the common bile duct, the pancreatic duct, the cystic duct, and the hepatic ducts.

11. The kit of claim 10, wherein the tool includes at least one of a fluid delivery device, a fluid removal device, a laser, a basket, a brush, a blade, a balloon dilator, a balloon, a catheter, a stent, a fiber optic viewing device, and combinations thereof.

12. The kit of claim 10, wherein the tool is movable along the lumen of the tubular body of the endoscope.

13. The kit of claim 10, wherein the tool includes at least one of a groove and a protrusion such that the tool is engageable with and movable along the lumen of the tubular body of the endoscope.

14. The kit of claim 10, wherein the lumen of the tubular body of the endoscope includes at least one of a groove and a protrusion to engage at least a portion of the tool and to allow the tool to be manipulated by manipulation of the endoscope.

15. An apparatus for performing bile or pancreatic duct endoscopy upon a patient having a mouth and an ampulla of Vater, the apparatus comprising:
a wire adapted to extend from the patient's mouth to the patient's ampulla of Vater;
an endoscope comprising a generally tubular body, the tubular body including a lumen and an outer diameter, the outer diameter being less than about 7 mm, the lumen dimensioned to receive the wire such that the endoscope is positioned over and is movable along the wire; and
an overtube having a lumen dimensioned to receive the endoscope, the overtube being positioned over the endoscope and movable along the wire.

16. The apparatus of claim 15, wherein the outer diameter of the tubular body of the endoscope is greater than about 3.4 mm.

17. The apparatus of claim 15, wherein the overtube has an inner diameter sufficiently large to permit movement of the endoscope through the overtube and along the wire.

18. The apparatus of claim 15, wherein:
the patient has a stomach;
the endoscope is adapted to be positioned at least as distally as the patient's ampulla of Vater; and
the overtube is movable into a position proximate the patient's stomach.

19. The apparatus of claim 15, wherein the overtube is positioned at least partially within the patient's stomach to facilitate movement of the endoscope along the wire within the patient's stomach.

20. The apparatus of claim 15, further comprising a tool dimensioned to be positioned within and movable along the lumen of the tubular body of the endoscope, the tool adapted to perform at least one of diagnosis and therapy in at least one of the ampulla of Vater, the common bile duct, the pancreatic duct, the cystic duct, and the hepatic ducts.

21. The apparatus of claim 20, wherein the tool includes at least one of a fluid delivery device, a fluid removal device, a laser, a basket, a brush, a blade, a balloon dilator, a balloon, a catheter, a stent, a fiber optic viewing device, and combinations thereof.

22. A method for performing endoscopy of a bile or pancreatic duct of a patient, the method comprising:
navigating a wire perorally or pernasally to and within at least one of the ampulla of Vater, the common bile duct, the pancreatic duct, the cystic duct, and the hepatic ducts of the patient;
providing an endoscope, the endoscope comprising a generally tubular body, the tubular body including a lumen and an outer diameter, the outer diameter being less than about 7 mm;
installing the endoscope over the wire while the wire is in place within at least one of the ampulla of Vater, the common bile duct, the pancreatic duct, the cystic duct, and the hepatic ducts of the patient such that the wire is received within the lumen of the tubular body of the endoscope;
navigating the endoscope along the wire to a position within at least one of the ampulla of Vater, the common bile duct, the pancreatic duct, the cystic duct, and the hepatic ducts; and
viewing the at least one of the ampulla of Vater, the common bile duct, the pancreatic duct, the cystic duct, and the hepatic ducts with the endoscope.

23. The method of claim 22, wherein the endoscope is a first endoscope, the method further comprising navigating a second endoscope perorally or pernasally to the ampulla of Vater, the second endoscope comprising a generally tubular body having a larger diameter than that of the first endoscope.

24. The method of claim 23, wherein:
the wire is dimensioned to be received within and movable along a lumen of the second endoscope; and
navigating the wire includes navigating the wire along the second lumen.

25. The method of claim 22, further comprising removing the wire from the at least one of the ampulla of Vater, the common bile duct, the pancreatic duct, the cystic duct, and the hepatic ducts of the patient by moving the wire relative to the lumen of the tubular body of the endoscope.

26. The method of claim 22, further comprising navigating a tool along the lumen of the endoscope to a position within at least one of the ampulla of Vater, the common bile duct, the pancreatic duct, the cystic duct, and the hepatic ducts.

27. The method of claim 26, further comprising performing at least one of diagnosis and therapy with the tool.

28. The method of claim 26, further comprising performing at least one of fluid delivery, fluid removal, cauterization, lithotripsy, tissue removal, stone removal, biopsy, surgery, stenting, stone reduction, and combinations thereof with the tool.

29. The method of claim 22, further comprising:
providing an overtube having a lumen dimensioned to receive the endoscope and the wire; and
moving the overtube along the wire to position the overtube within the patient.

30. The method of claim 29, wherein navigating the endoscope along the wire includes navigating the endoscope within the lumen of the overtube.
